(19) United States
(12) Patent Application Publication
(10) Pub. No.: US 2010/0286573 A1
(43) Pub. Date: Nov. 11, 2010

(54) SYSTEM AND METHODS OF TREATMENT USING ULTRA-WIDEBAND, HIGH POWERED FOCUSING EMITTERS

(75) Inventor: Karl H. Schoenbach, Norfolk, VA (US)

Correspondence Address: Gregory A. Nelson;Novak Druce & Quigg LLP, 525 Okeechobee Blvd, Suite 1500, West Palm Beach, FL, 33401, US

(73) Assignee: Old Dominion University Research Foundation, Norfolk, VA (US)

(21) Appl. No.: 12/681,539

(22) PCT Filed: Oct. 3, 2008
(86) PCT No.: PCT/US08/78714

§ 371 Date: Jul. 27, 2010

Related U.S. Application Data
(60) Provisional application No. 60/978,035, filed on Oct. 5, 2007.

(51) Int. Cl. A61N 7/02

(52) U.S. Cl. 601/3

(57) ABSTRACT

A method of inducing local hyperthermia in patient tissue is provided. The method includes generating ultra-wide-band radiation, conveying the ultra-wideband radiation to a focusing element, and focusing the ultra-wideband radiation on a target with the focusing element. A system for inducing local hyperthermia in patient tissue is also provided. The system includes a power source for generating ultra-wideband radiation, and a focusing element for focusing the ultra-wideband radiation on a target.

### RESEARCH OR DEVELOPMENT

The United States Government may have certain rights in the invention by virtue of research support provided by the United States Air Force Office of Scientific Research (AFOSR).

### FIELD OF THE INVENTION

The present invention is related to the field of patient treatments, and more particularly, to techniques for treating patients using hyperthermia.

### BACKGROUND OF THE INVENTION

Hyperthermia can be used as a form of treatment in which body tissue is exposed to high temperatures. Hyperthermia has been used, for example, to damage or kill cancer cells and to make cancer cells more sensitive to collateral modes of treatment. Different forms of energy can be used to induce hyperthermia, including microwave, radio wave, and ultrasound.

In treating tumors or other cancers, an optimum effect can be achieved if the tumor is locally heated such that healthy tissue remains unaffected. The treatment can be effected through external approaches, such as that undertaken for treating skin cancer. An external approach to treating skin cancer, for example, utilizes microwaves with slight tissue penetration depth to induce hyperthermia in melanoma tumors.

For treating deeper lying tumors, heat sources can be inserted directly into a tumor to induce hyperthermia. A procedure known as radio frequency ablation (RFA) utilizes radio frequency radiation, which is applied to the tumor. Radio frequency also has been used to treat deeper lying tumors and cancerous tissues, but typically only for regional hyperthermia. According to this technique large volumes of tissue are usually heated by placing external applications around that portion of a patient's body that is to be treated.

Conventionally, if local heating of a tumor or part of a tumor is to be undertaken, short-wavelength radiation can be used, the tissue penetration depth being relatively slight. An antenna can be used to transfer the energy from the source to the tumor. Because of the small penetration depth, this technique can be effective for skin cancers, but typically can not be used to effectively treat deeper lying tissue. Alternatively, for deeper penetration, an interstitial technique can be used, according to which a probe is inserted into the tumor in order to induce hyperthermia by transfer of energy from the tip of the probe to the tumor.

### SUMMARY OF THE INVENTION

The present invention is directed to systems and methods for inducing local hyperthermia in tissue using ultra-wideband radiation. In one aspect of the invention, local hyperthermia is induced by generating ultra-wideband radiation and focusing the radiation using high-power focusing emitters. In another aspect of the invention, local hyperthermia is induced by generating ultra-wideband radiation and focusing the radiation using certain types of lenses.

### BRIEF DESCRIPTION OF THE DRAWINGS

There are shown in the drawings, embodiments which are presently preferred. It is expressly noted, however, that the invention is not limited to the precise arrangements and instrumentalities shown.

FIG. 1 is a schematic diagram of a system for generating local hyperthermia in tissue, according to one embodiment of the invention.

FIG. 2 is a plot of the electric field distribution that can be obtained using the system of FIG. 1

FIG. 3 is a flowchart of exemplary steps in a method for generating local hyperthermia in tissue, according another embodiment of the invention.

### DETAILED DESCRIPTION

The invention is directed to systems and methods for treating a patient using ultra-wideband radiation. By utilizing ultra-wideband radiation it is possible to induce local heating with external sources, not only on the surface of the patient's body, but in deeper lying tissue as well.

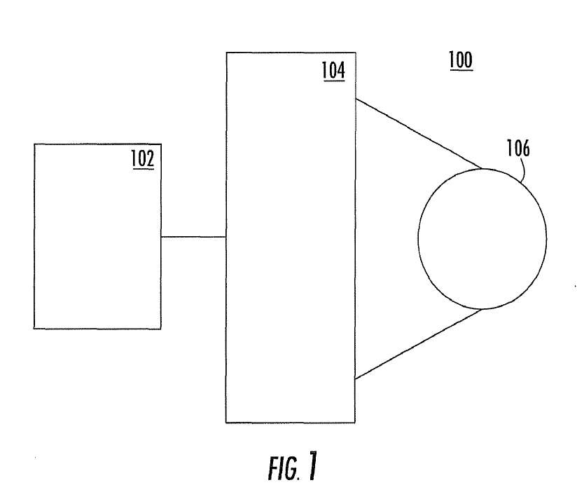

Referring initially to FIG. 1, a system 100 according to one embodiment of the invention is schematically illustrated. The system 100 illustratively includes an energy source 102 for generating ultra-wideband radiation. The system 100 further illustratively includes a focusing element 104 for focusing the radiation generated by the energy source 102 on a target 106. The target 106, more particularly, can be a tumor identified in the body of patient. As explained below, however, in alternative uses, the system 100 can be used for other purposes, including treating a subject for corrective or cosmetic reasons.

The focusing element 104 can comprise one or more prolate spheroidal reflectors for focusing the radiation generated by the energy source 102. In an alternate embodiment, the focusing element 104 can comprise a lens for focusing the radiation on the target 106.

Operatively, the system 100 generates ultra-short electrical pulses. By using ultra-short electrical pulses, preferably with rise times on the order of less than 1 nanosecond, it is possible to focus the energy into a small volume with characteristic dimensions of millimeters. Since the frequency distribution for the electromagnetic waves that correlate to such ultra-short pulses—even for 100 ps pulses—reaches only to a few GHz, the penetration depth of the radiation exceeds that of millimeter wave radiation. Indeed, the penetration depth obtained using ultra-short pulses can considerably exceed that obtained with millimeter wave radiation. It is, therefore, possible to reach tumors located more deeply in a patient's body with this external approach, rather than using probes that must be inserted into the patient.

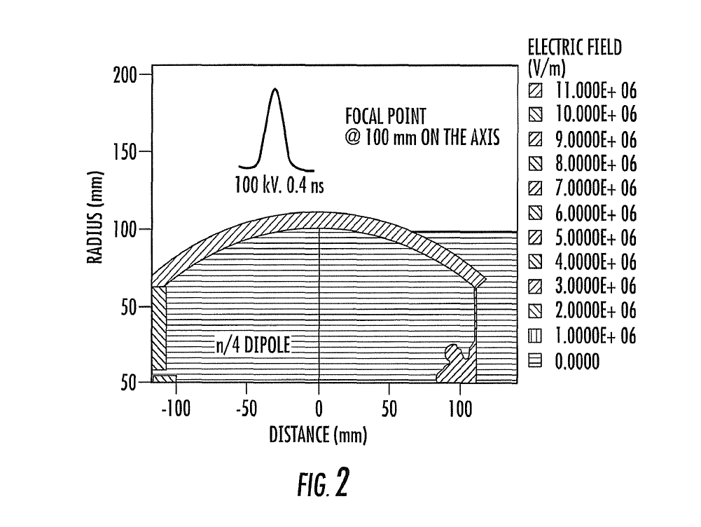

FIG. 2 provides a plot showing this effect. In particular, FIG. 2 shows the computed electric field distribution indicating the large values of electric field in the focal volume at a 100 mm distance. The focusing effect can be achieved, according to one embodiment, if the focusing element 104 comprises an oblate spheroidal reflector, which focuses the energy emitted from an antenna in the left focal volume to that in the right focal volume. Alternatively, instead of reflectors, the focusing element 104 can comprise lenses configured to obtain similar focusing effects.

The energy distribution, which corresponds to the temperature distribution, scales with the square of the electric field intensity. Correspondingly, the volume where critical temperatures are reached is even smaller than that characterized by the electric field value. It is known that tumor tissue generally has a higher conductivity than healthy tissue. See, e.g. Kenneth R. Foster and Hermann P. Schwan, “Dielectric Properties of Tissues” in Handbook of Biological Effects of Electromagnetic Fields, CRC Press, 1995, page 68 ff), the disclosure of which is hereby incorporated by reference. Thus, the effect of the electromagnetic field in the tumor is even more amplified. The energy can be focused into a well defined volume, if ultra-short pulses are used, according to the invention.

As will be understood by one of ordinary skill in the art, localized hyperthermia can cause apoptosis of tumor cells. This connection of hyperthermia with apoptosis has been demonstrated in several studies. Mild hyperthermia also has been shown to increase the sensitivity of tumors to other agents. Accordingly, a combination therapy, where local heating is combined with another procedure (e.g., ionizing radiation therapy or local administration of toxins, such as cisplatinum or bleomycin) may increase the probability for tumor reduction. See, e.g., International Journal of Oncology, 2007 Apr., 30(4):841-7, the disclosure of which is hereby incorporated by reference.

The use of hyperthermia, generated locally by means of the exemplary system 100, or a similar ultra-wideband imaging system according to the invention, is not restricted to the elimination of tumor cells. Other unwanted tissue, such as adipose tissue, can also be affected by hyperthermia. Focusing the ultra-wideband radiation into fat tissue is likely to cause apoptosis of fat cells. Since through the focusing effect, the energy density at the skin would be minimized, this treatment can affect only non-wanted tissue, without adversely affecting a patient's skin.

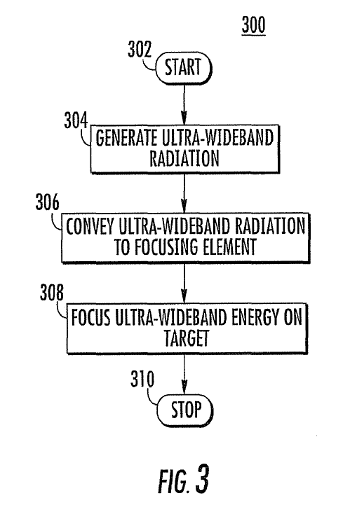

FIG. 3 is a flowchart of exemplary steps of a method 300 for inducing local hyperthermia, according to another embodiment of the invention. The method 300, after starting at step 302, includes generating ultra-wideband radiation at step 304. At step 306, the ultra-wideband radiation thus generated is conveyed to a focusing element. Using the focusing element, the ultra-wideband radiation is focused on a target, such as a tumor, at 308. The method illustratively concludes at step 310.

In still another embodiment, however, treatment of a patient includes the inducement of local hyperthermia in conjunction with an additional therapy. The additional therapy, according to one embodiment comprises ionizing radiation therapy. In an alternative embodiment, the additional therapy comprises the local administration of a toxin. The toxin can be, for example, cisplatinum, bleomycin, or similar such toxin.

### EXAMPLE

A calculation of the temperature, obtainable with such an ultra-wideband focusing system is shown in the following. Based on modeling results similar to those shown in FIG. 2, the electric field in the focal volume can reach values of more than 100 kV/cm. With a pulse duration of 100 ps, the energy density in the focal point (assuming a resistivity of the tissue of 50 Ohm cm) is 0.02 Joule per cubic centimeter. This corresponds to a temperature increase of approximately 0.005 degree K per pulse. Using a pulse power system which generates the required electric fields at a repetition rate of 10 kHz, which is technically feasible, the local temperature increase in the tissue after 1 second exposure would be 50 degree K. This simple calculation does not take into account thermal loss processes, such as thermal conduction, which would cause a reduction in the peak energy, but even when we include such loss processes, the temperature locally would exceed that required for apoptosis induction considerably.

The use of ultra-wideband radiation at extreme power levels opens a new avenue of radiation therapy apart from ionizing radiation. Focusing the ultra-wideband radiation into small volumes, generates high temperature locally within a patient's body, where it is needed, but has only minor effects on neighboring tissue.

By using ultrawideband radiation, the present disclosure can overcome the obstacles of presently used methods. For instance, the exemplary embodiments can generate local heating with external sources not just on the surface but also for deeper lying tissue. This technique of the exemplary embodiments is based on the focusing effect of prolate spheroidal reflectors or certain types of lenses.

The foregoing description of preferred embodiments of the invention have been presented for the purposes of illustration. The description is not intended to limit the invention to the precise forms disclosed. Indeed, modifications and variations will be readily apparent from the foregoing description. Accordingly, it is intended that the scope of the invention not be limited by the detailed description provided herein.

1. A system for inducing local hyperthermia in patient tissue, the system comprising:
a power source for generating ultra-wideband radiation; and
a focusing element for focusing the ultra-wideband radiation on a target.

2. The system of claim 1, wherein the focusing element comprises at least one prolate spheroidal reflector.

3. The system of claim 1, wherein the focusing element comprises at least one focusing lens.

4. The system of claim 1, wherein the ultra-wideband radiation is an ultra-short electrical pulse.

5. The system of claim 4, wherein the ultra-short electrical pulse has a rise time of less than one (1) nanosecond.

6. A method for inducing local hyperthermia in patient tissue, the method comprising:
generating ultra-wideband radiation;
conveying the ultra-wideband radiation to a focusing element; and
a focusing the ultra-wideband radiation on a target with the focusing element.

7. The method of claim 6, wherein the focusing element comprises at least one prolate spheroidal reflector.

8. The method of claim 6, wherein the focusing element comprises at least one focusing lens.

9. The method of claim 6, wherein the ultra-wideband radiation is an ultra-short electrical pulse.

10. The method of claim 9, wherein the ultra-short electrical pulse has a rise time of less than one (1) nanosecond.

11. A method of treating biological tissue, the method comprising:
focusing ultra-wideband radiation on the biological tissue; and
subsequently treating the tissue with another mode of treatment.

12. The method of claim 11, wherein the other mode of treatment comprises ionizing radiation.

13. The method of claim 11, wherein the other mode of treatment comprises the local application of a toxin.

14. The method of claim 13, wherein the toxin is cisplatinum.

15. The method of claim 13, wherein the toxin is bleomycin.

16. The method of claim 11, wherein focusing comprises focusing the ultra-wideband radiation with at least one prolate spheroidal reflector.

17. The method of claim 11, wherein focusing comprises focusing the ultra-wideband radiation with at least one focusing lens.

18. The method of claim 11, wherein the ultra-wideband radiation is an ultra-short electrical pulse.

19. The method of claim 11, wherein the ultra-short electrical pulse has a rise time of less than one (1) nanosecond.
